(19) United States
(12) Patent Application Publication
(10) Pub. No.: US 2013/0150265 A1
(43) Pub. Date: Jun. 13, 2013

(54) CHEMICAL SYNTHESIS USING UP-CONVERTING PHOSPHOR TECHNOLOGY AND HIGH SPEED FLOW CYTOMETRY

(71) Applicants: Robert Balog, Sunnyvale, CA (US); David E. Cooper, Palo Alto, CA (US); Steven Crouch-Baker, Palo Alto, CA (US); Alexander J. Hallock, Redwood City, CA (US); Georgina Hum, Menlo Park, CA (US); Maheen Samad, Mountain View, CA (US); Angel Sanjurjo, San Jose, CA (US)

(72) Inventors: Robert Balog, Sunnyvale, CA (US); David E. Cooper, Palo Alto, CA (US); Steven Crouch-Baker, Palo Alto, CA (US); Alexander J. Hallock, Redwood City, CA (US); Georgina Hum, Menlo Park, CA (US); Maheen Samad, Mountain View, CA (US); Angel Sanjurjo, San Jose, CA (US)

(21) Appl. No.: 13/578,177

(22) PCT Filed: Feb. 9, 2011
(86) PCT No.: PCT/US2011/024203

§ 371 Date: Nov. 15, 2012

Related U.S. Application Data
(60) Provisional application No. 61/302,863, filed on Feb. 9, 2010.
(60) Provisional application No. 61/407,370, filed on Oct. 27, 2010.

(51) Int. Cl. C07H 21/04; C07K 1/04

(52) U.S. Cl. CPC C07H 21/04; C07K 1/047
USPC 506/30; 506/43

(57) ABSTRACT

The invention offers the ability to rapidly synthesize multiple chemical compounds, particularly polymers of varying sequences, in parallel on the surfaces of carrier beads. Tinvention involves attaching up-converting phosphors (UCP's) to beads to create up-converting phosphor-loaded beads (UCP-loaded beads) with unique spectral characteristics. Using a dynamic sorting architecture each bead is cataloged based on its spectral characteristics, assigned a compound or polymer to be synthesized, and subjected to multiple rounds of sorting by a flow cytometer, wherein each round sorts the bead to an appropriate bin for a selected chemical reaction, such as the attachment of a monomeric subunit of the polymer sequence.

### CROSS REFERENCE TO RELATED APPLICATION

This application claims benefit of priority to PCT International Application No. PCT/US2011/024203, filed 9 Feb. 2011; which claims benefit to U.S. Provisional Applications 61/302,863, filed 9 Feb. 2010; and 61/407,370, filed 27 Oct. 2010, which are incorporated herein by reference.

### TECHNOLOGICAL FIELD

This technology generally relates to a large parallel memory architecture and high speed query scheme to access a large and potentially independent set of sort sequences for steering uniquely tagged elements through a high speed element sorter. Specifically, the sorter can be a flow cytometer and the elements can be up-converted phosphor-loaded beads.

### BACKGROUND

The field of synthetic biology is in its infancy; however the field has the potential to radically change how biological systems are engineered. In just the last few years researchers have chemically synthesized a poliovirus^{[1]}, bacteriophage^{[2]}, mycoplasma gentialium genome^{[3]}, engineered organisms to produce anti-malarial drug precursors^{[4]}, and determined the minimal working genomes of selected bacteria^{[5, 6]}. Currently, however, experiments in synthetic biology are conducted only in specialized laboratories by extensively trained scientists. New tools that allow for the rapid, cost effective synthesis of nucleic acids must be developed to move synthetic biology from specialized laboratories to general use.

The cost of DNA sequencing has dropped dramatically in recent years. For example, the Human Genome Project spent about $1,000,000,000 to sequence the human genome. Today, human genome sequencing costs about $50,000. By contrast, DNA synthesis is still relatively expensive; therefore the cost synthesizing numerous sets of DNA sequences, such as those used in microarrays, can be prohibitive. Thus, decreased expense and reduced technical difficulty of synthesizing nucleic acid sequences that the method of the invention provides will benefit society in ways beyond the development of synthetic genomes.

In particular, microarray technology provides the means to assess the expression of hundreds, or even thousands of individual genes at one time. In order to accomplish such a feat, a microarray may need to comprise thousands, or even millions, of individual DNA sequences. Current technologies for DNA microarray analysis fall into either of two primary categories: (1) a serial fabrication process, which has low material and equipment costs to perform; or (2) a parallel fabrication process, which has high material and equipment costs. Each fabrication process takes weeks to months to fabricate an entirely new high-density array.

The current industry standards for the serial fabrication process include spotted arrays (sequences spotted on a planar substrate) and bead-based arrays (sequences coupled to a uniquely labeled bead). The company, Illumina, manufactures microarrays based on serial fabrication approaches. Typically, microarray companies like Illumina individually synthesize unique DNA sequences using a serial process involving standard DNA phosphoramidite chemistry. Serial approaches to array fabrication require significant labor to synthesize each oligonucleotide sequence in a separate reaction, and couple each sequence to a substrate. Because each DNA probe is synthesized in series, the time required to fabricate an array scales with the number of probes in the DNA microarray.

The industry standard for parallel fabrication of oligonucleotides for microarrays is a photolithographic synthesis approach that requires expensive photoliable phosphoramidites. Spatial separation of the probes and the photolithographically defined removal of the photoprotecting groups allows for precise control of when and where each DNA base is added. Because oligonucleotides can be fabricated in parallel, the time required to fabricate a DNA microarray is independent of the number of different probes and depends only on the length of the probes. For example, the synthesis of 1 million 50-base probes requires no more than 200 synthesis steps (50×4, the number of bases in the probe times the number of DNA bases). The company, Affymetrix is a leader in the fabrication of microarrays by a photolithographic synthesis approach.

Unlike the foregoing fabrication processes, the method of the invention provides a novel low-cost platform for the parallel fabrication of multiple polymers of varying monomeric subunit sequences, e.g., oligonucleotides. For example, the method of the invention can fabricate DNA microarrays with more than one million different probe sequences in less than one day, and a hundred times cheaper than currently possible. These advances in polymer fabrication that are brought by the method of the invention capitalize on combining up-converting phosphor technology (UPT) and recent advances in flow cytometry technology to allow low-cost, rapid synthesis of DNA microarrays using traditional DNA synthesis chemicals

### SUMMARY OF THE INVENTION

The method of the invention offers the ability to rapidly synthesize multiple chemical compounds, particularly polymers of varying sequences, in parallel on the surfaces of carrier beads. More specifically, the invention involves attaching up-converting phosphors (UCP's) to beads to create up-converting phosphor-loaded beads (UCP-loaded beads) with unique spectral characteristics. Using a dynamic sorting architecture each bead is cataloged based on its spectral characteristics, assigned a compound or polymer to be synthesized, and subjected to multiple rounds of sorting by a flow cytometer, wherein each round sorts the bead to an appropriate bin for a selected chemical reaction, such as the attachment of a monomeric subunit of the polymer sequence.

The up-converting phosphors used in the invention are rare-earth doped ceramic materials with the unique property of emitting a single higher energy visible photon upon excitation with two lower energy near-infrared photons. The combination of different absorber and emitter rare earth ions, ion doping levels, and the use of different crystal host materials allows for the synthesis of different spectrally unique phosphor compositions that can be excited with a single, highly efficient laser. Another useful characteristic of up-converting phosphors is that they can be detected with high sensitivity; a single, efficient gallium arsenide (GaAs) semiconductor laser provides sufficient excitation power to enable the detection of single phosphor particles, each of which emits in a narrow spectral band facilitating multiplexing. The emission intensity is proportional to the number of UCP particles in a sample; therefore, the emission signals represent a quantitative measurement. Detection of up-converting phosphor emission wavelengths are also intrinsically background free because the two-photon up-conversion process is not observed in naturally occurring materials. Furthermore, the long wavelengths that are used to excite up-converting phosphors produce minimal background interference due to significantly reduced or eliminated autofluorescence. A permanent record of up-converting phosphors' spectral characteristics is also possible to attain because the solid-state nonradiative transfer process between rare earth ions does not photobleach. Up-converting phosphor particles can up convert IR light after many years on a laboratory bench.

In one embodiment, the invention relates to a carrier bead having a generally spherical shape and a layer of at least one up-converting phosphor particle on the bead's surface. By attaching different phosphor particles in different quantities and ratios to the surface of the carrier bead, the up-converting phosphor-loaded bead can act as a “communicating optical pipe.” This design assures that all the phosphor particles are available and respond in a predictable way to the incoming light and emit to the same environment. A simple version of the composite phosphor is illustrated in FIG. 2. By changing the composition and concentrations of the up-converting phosphors on the surface of each bead, the method of the invention obtain a large number of particles with distinguishable spectral emissions. The particle size will be mostly dictated by the original bead size, and the degree of loading and final external coating (typically SiO₂). The index of refraction of the core can be tailored to obtain maximum efficiency.

In one embodiment, the invention provides a method of synthesizing at least two polymers by a stepwise combination of monomeric units. The method comprises the steps of:

a) obtaining at least two sets of UCP-loaded beads, wherein the UCP-loaded beads within each set are spherical beads with a layer of at least one up-converting phosphor particle on the bead surface and each set has a unique excitation or emission identity;

b) detecting the emission properties of the UCP-loaded beads using a computer system-controlled flow cytometer;

c) recording the emission properties of each set of UCP-loaded beads to a database located on a computer-readable medium using the computer-controlled flow cytometer, wherein the database assigns each unique UCP-loaded identity to a specified polymer sequence;

d) sorting the UCP-loaded beads into any one of a number of bins by sets, wherein each bin is correlated with a specified monomeric subunit, and wherein the assignment of each UCP-loaded bead to a bin is based on the first monomeric subunit of the polymer sequence that is assigned to the UCP-loaded bead in the database of step (c);

e) attaching the monomeric subunits within each bin to the surfaces of the UCP-loaded beads sorted to the bin;

f) pooling the UCP-loaded bead sets after completion of step (e)

g) optionally re-sorting the UCP-loaded beads from step (f) into bins using the computer-controlled flow cytometer, wherein the UCP-loaded beads' spectral identities are detected, and each UCP-loaded bead is sorted to a bin according to the next monomeric subunit to be added to the polymer sequence assigned to each UCP-loaded bead set in the database of step (c);

h) reacting the UCP-loaded beads under conditions sufficient to attach a selected monomeric subunit to the most-recently attached monomeric subunit;

i) pooling the UCP-loaded beads after completion of step (h);

j) repeating steps (g)-(i) to produce a desired polymer on each set of UCP-loaded beads; and

k) optionally cleaving a polymer from its UCP-loaded bead. Variations and preferred embodiments of this method are described below.

### BRIEF DESCRIPTION OF THE DRAWINGS

FIG. 1 shows: (a) a diagram of the two-1R-photon transfer from an Yb ion absorber to the Er ion emitter and the subsequent emission of a single visible photon; and (b) emission spectra for 9 different compositions.

FIG. 2 shows a graphic description of a method for creating millions of spectrally unique UCP-loaded beads. In particular, the figure graphically depicts twelve spectrally unique 75 nm UCP particles (UCP Particle Spectra-(1-12)), as well as depictions of three UCP-loaded beads, which are each 10-μm silica core beads on which thousands of individual UCP particles are fused.

FIG. 3 shows a graphical representation of the first two steps necessary to fabricate a DNA microarray on the UCP-loaded beads. The first step of the process is to determine what sequence will be added to each spectrally unique UCP-loaded bead. The flow cytometer is then used to sort the beads into bins based on the first base in the desired sequence. Next, the base is added and the beads are re-pooled. The end result is a pool of UCP-loaded beads with the first base in the desired sequence added. Next, the UCP-loaded beads are resorted, based on the second base in the desired sequence for each bead. The appropriate base is added to all the beads in each bin, and then the beads are re-pooled for the third step. The process is repeated until the desired sequence is fully synthesized on each UCP-loaded bead.

FIG. 4 shows a graphic representation of a dynamic sorting architecture example for DNA synthesis.

FIG. 5 shows a more detailed graphical representation of the initial catalog run that FIG. 4 depicts.

FIG. 6 shows a more detailed graphical representation of the first run (Round 1) that FIG. 4 depicts.

FIG. 7 shows a more detailed graphical representation of the second run (Round 2) that FIG. 4 depicts.

FIG. 8 shows a graphical representation of the Dynamic Sorting Architecture (DSA) and the relationship between the high speed performance of the sorting method to the unit time window that enables that high speed. The DSA performs UCP-loaded bead identification, database query, and sort decision functions in immediate succession.

FIG. 9 shows approximately 6 to 6.5 micron diameter silica particles were coated with YYbEr particles.

FIG. 10 shows approximately 6 to 6.5 micron diameter silica particles were coated with coated with three different up-converting phosphor particles.

### DETAILED DESCRIPTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the invention, the following terms are defined below.

The invention involves a unique combination of up-converting phosphor and flow cytometric technologies to be used in chemical synthesis. The invention relates to a novel platform that allows for the rapid, low-cost, parallel custom synthesis of compounds and polymers. The invention also relates to carrier beads that are coated with up-converted phosphor particles (up-converted phosphor-loaded carrier beads) and used in the methods of the invention.

In one embodiment a method of the invention synthesizes at least two polymers by a stepwise or a sequential combination of monomeric units. The method comprises the steps of:

a) obtaining at least two sets of UCP-loaded beads, wherein the UCP-loaded beads within each set are spherical beads with a layer of at least one up-converting phosphor particle on the bead surface and each set has a unique excitation or emission identity;

b) detecting the emission properties of the UCP-loaded beads using a computer system-controlled flow cytometer;

c) recording the emission properties of each set of UCP-loaded beads to a database located on a computer-readable medium using the computer-controlled flow cytometer, wherein the database assigns each unique UCP-loaded identity to a specified polymer sequence;

d) sorting the UCP-loaded beads into any one of a number of bins by sets, wherein each bin is correlated with a specified monomeric subunit, and wherein the assignment of each UCP-loaded bead to a bin is based on the first monomeric subunit of the polymer sequence that is assigned to the UCP-loaded bead in the database of step (c);

e) attaching the monomeric subunits within each bin to the surfaces of the UCP-loaded beads sorted to the bin;

f) pooling the UCP-loaded bead sets after completion of step (e)

g) optionally re-sorting the UCP-loaded beads from step (f) into bins using the computer-controlled flow cytometer, wherein the UCP-loaded beads' spectral identities are detected, and each UCP-loaded bead is sorted to a bin according to the next monomeric subunit to be added to the polymer sequence assigned to each UCP-loaded bead set in the database of step (c);

h) reacting the UCP-loaded beads under conditions sufficient to attach a selected monomeric subunit to the most-recently attached monomeric subunit;

i) pooling the UCP-loaded beads after completion of step (h);

j) repeating steps (g)-(i) to produce a desired polymer on each set of UCP-loaded beads; and

k) optionally cleaving a polymer from its UCP-loaded bead.

Another method of the invention for synthesizing at least two polymers by a stepwise or sequential combination of monomers comprises the steps of:

a) providing at least two sets of UCP-loaded beads, wherein the UCP-loaded beads within each set are spherical beads with a layer of at least one up-converting phosphor particle on the bead surface and each set has a unique excitation or emission identity;

b) optionally attaching a monomeric subunit to the at least two sets of UCP-loaded beads;

c) detecting the emission properties of the UCP-loaded beads using a computer system-controlled flow cytometer;

d) recording the emission properties of each set of UCP-loaded beads to a database located on a computer-readable medium using the computer-controlled flow cytometer, wherein the database assigns each unique UCP-loaded identity to a specified polymer sequence;

e) sorting the UCP-loaded beads into any one of a number of bins by sets, wherein each bin is correlated with a specified monomeric subunit, and wherein the assignment of each UCP-loaded bead to a bin is based on the first monomeric subunit of the polymer sequence that is assigned to the UCP-loaded bead in the database of step (d);

f) attaching the monomeric subunits within each bin to the surfaces of the UCP-loaded beads sorted to the bin;

g) pooling the UCP-loaded bead sets after completion of step (f);

h) optionally re-sorting the UCP-loaded beads from step (g) into bins using the computer-controlled flow cytometer, wherein the UCP-loaded beads' spectral identities are detected, and each UCP-loaded bead is sorted to a bin according to the next monomeric subunit to be added to the polymer sequence assigned to each UCP-loaded bead set in the database of step (d);

i) reacting the UCP-loaded beads under conditions sufficient to attach a selected monomeric subunit to the most-recently attached monomeric subunit;

j) pooling the UCP-loaded beads after completion of step (i);

k) repeating steps (h)-(j) to produce a desired polymer on each set of UCP-loaded beads;

l) optionally cleaving a polymer from its UCP-loaded bead.

In another general embodiment, a method of the invention provides a stepwise or sequential chemical synthesis comprising the steps of:

a) providing at least two sets of UCP-loaded beads, wherein the UCP-loaded beads within each set are spherical beads with a layer of at least one up-converting phosphor particle on the bead surface and each set has a unique excitation or emission identity;

b) optionally attaching a first reactant to the at least two sets of UCP-loaded beads;

c) optionally detecting the emission properties of the UCP-loaded beads using a computer system-controlled flow cytometer;

d) recording the emission properties of each set of UCP-loaded beads to a database located on a computer-readable medium using the computer-controlled flow cytometer, wherein the database assigns each unique UCP-loaded identity to a specified product to be synthesized;

e) sorting the UCP-loaded beads into any one of a number of bins by sets, wherein each bin is correlated with a specified sequenced reaction step;

f) reacting the sorted UCP-loaded beads in step (e) according to the specified sequenced reaction step;

g) pooling the UCP-loaded bead sets after completion of step (f);

h) optionally re-sorting the UCP-loaded beads from step (g) into bins using the computer-controlled flow cytometer, wherein the UCP-loaded beads' spectral identities are detected, and each UCP-loaded bead is sorted to a bin according to the next sequential reaction step assigned to each UCP-loaded bead set in the database of step (d);

i) reacting the UCP-loaded beads in step (g) or step (h) according to the specified sequenced reaction step;

j) repeating steps (g)-(i) to synthesize a desired compound on each set of UCP-loaded beads; and

k) optionally cleaving a desired compound from its UCP-loaded bead.

As can be understood, the methods of the invention may be used for the parallel preparation of chemical compounds, particularly polymers and more particularly in the preparation of DNA, proteins, and other such polymeric sequences where the preparative steps or chemical reactions proceed in a stepwise fashion. An advantage of the methods is the ability to rapidly synthesize multiple chemical compounds in parallel using a combination of flow cytometry techniques and up-convertng phosphor technology. The various aspects of the methods of the invention, the steps and components, are discussed below.

Carrier Beads

A carrier bead of the invention has a generally spherical shape and a layer of at least one up-converting phosphor particle on the bead's surface. The carrier beads of the invention are termed up-converting phosphor-loaded beads or UCP-loaded beads. Any material that a practitioner would know to be appropriate for flow cytometry is suitable making the carrier beads of the invention. Spherical carrier beads are generally preferred. For example, certain embodiments of the invention use beads of a ceramic, such as, but not limited to, silica (SiO₂) or a silica-ceramic. Other embodiments, however, may use beads composed of non-ceramic materials (e.g., a polymer material or a metal). Regardless of the composition of the bead core, the invention allows a practitioner to tailor the bead's index of refraction to obtain maximum efficiency. Indeed, in various embodiments of the invention, the carrier bead cores are transparent to excitation wavelengths of the up-converting phosphors, or to their emission wavelengths, or both. Alternatively, in other embodiments, the carrier bead core can be coated with a metallic layer which is placed in between the bead and the up-converting phosphor particle layer. The metallic layer may be used to reflect the excitation and/or emission radiation of the up-converting phosphor, thereby avoiding loss due to absorption in the carrier bead.

The carrier bead cores of the invention may also vary in diameter. While the practitioner may select carrier beads of any diameter that is appropriate to obtain the desired spectral characteristics of the up-converting phosphor-loaded beads, bead diameters are typically 20 μm or less. For example, in some embodiments, the carrier bead may be about 5 μm, or 10 μm, or 15 μm. A mixture of different sized carrier beads may be used to prepare sets of UCP-loaded beads for use in the methods of the invention. Different sized UCP-loaded bead sets provides a further discriminator, size, between the beads as they are used in the methods of the invention. For example, different sized UCP-loaded beads may be sorted by their up-convertng phosphor identity and/or their size.

Up-Converting Phosphors

Up-converting phosphor particles are known for their use in diagnostic assays. See, e.g., U.S. Pat. Nos. 5,043,265 and 6,159,686. This invention uses up-converting phosphors to uniquely label individual carrier beads that serve as substrate surfaces for the parallel fabrication of polymers comprising variable sequences of monomeric subunits. Generally, an up-converting phosphor or combinations of up-converting phosphors uniquely identify a bead, and by extension a particular polymer. The method of the invention relies on flow cytometry to identify the unique phosphor labels by illumination with an excitation radiation, followed by detection of the emission radiation.

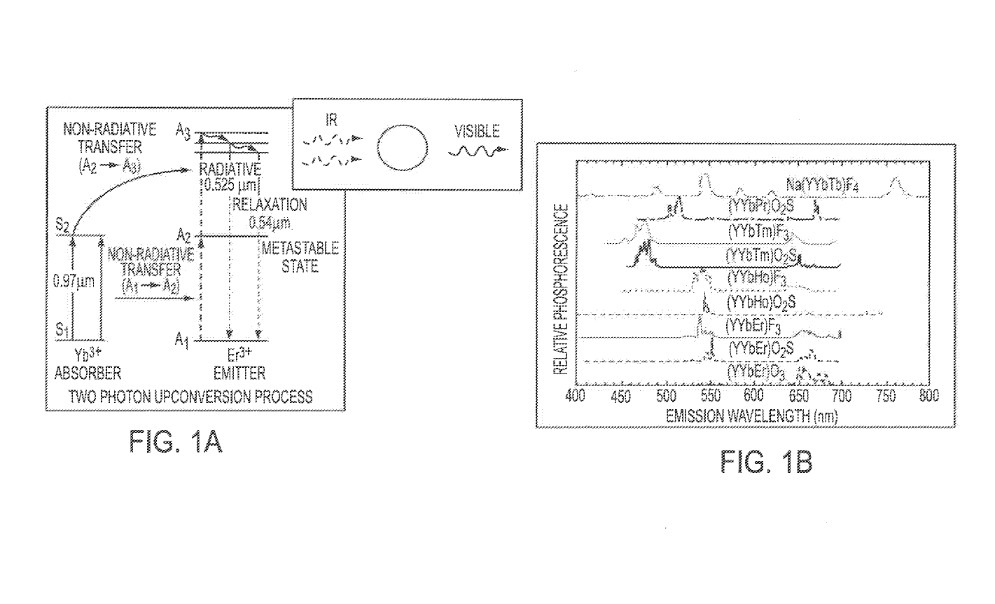

Up-converting phosphors are rare-earth doped ceramic materials with the unique property of emitting a single higher energy visible photon upon excitation with two lower energy near-infrared photons. Within the context of the invention, the notion of up-conversion refers to the emission of electromagnetic radiation at up-shifted frequencies (i.e., at higher frequencies than the excitation radiation). FIG. 1 diagrams the two-photon non-radiative transfer between two rare-earth ions doped together in a single up-converting phosphor particle resulting in emission of a single photon in the visible spectral region. The combination of different absorber and emitter rare earth ions, ion doping levels, and the use of different crystal host materials allows for the synthesis of different spectrally unique phosphor compositions. Up-converting phosphor particles also retain their emission characteristics at high temperatures. Thus, in various embodiments of the invention, up-converting phosphor particles can be doped into molten glass without losing their ability to up-convert.

As used herein, the terms “excitation,” “excitation wavelength,” and the like refer to an electromagnetic radiation wavelength that, when absorbed by an up-converting label, produces a detectable emission from the up-converting particle, wherein the emission is of a shorter wavelength (i.e., higher frequency radiation) than the particle's excitation wavelength.

As used herein, the term “emission,” “emission wavelength,” and the like refer to a wavelength that is emitted from an up-converting particle subsequent to, or contemporaneously with, illumination of the up-converting particle with one or more excitation wavelengths. Emission wavelengths of up-converting particles are shorter (i.e., higher frequency radiation) than the corresponding excitation wavelengths. Excitation properties and label emission wavelengths are unique to individual up-converting phosphor species, and are readily determined by performing simple excitation and emission scans. Some embodiments of the invention employ up-converting phosphor particles that are optimally excited by infrared radiation of about 950 to 1000 nm; for example, but not limited to, about 960 to 980 nm. In an exemplary embodiment of the invention, an up-converting phosphor with the formula YF₃:Yb_{0.10}E_{0.01} exhibits a luminescence intensity maximum at an excitation wavelength of about 980 nm. In various other embodiments of the invention, up-converting phosphors typically have emission maxima that are in the visible range.

Up-conversion has been found to occur in certain materials containing rare earth ions in certain crystal materials. For example, ytterbium and erbium act as an activator couple in a phosphor host material such as barium-yttrium-fluoride. The ytterbium ions act as the absorber, and transfer energy non-radiatively to excite the erbium ions. The emission is thus characteristic of the erbium ion's energy levels. U.S. Pat. No. 6,312,914 contains examples, and a discussion of the relationship of phosphor hosts and activator couples, and U.S. Pat. No. 6,159,686 is discusses using up-converting phosphor particles to detect molecules, including analytes, and biological macromolecules.

Up-converting phosphor particles are known materials. The up-converting phosphors can be manufactured as described in various published methods, including but not limited to the following: U.S. Pat. No. 6,312,914; Yocom et al., (1971) Metallurgical Transactions 2: 763; Kano et al., (1972). J. Electrochern. Soc., p. 1561; Wittke et al. (1972) J. Appl. Physics 43:595; Van Uitert et al. (1969) Mat. Res. Bull. 4: 381; which are incorporated herein by reference. Other references which may be referred to are: Jouart J. P. and Mary G. (1990) J. Luminescence 46: 39; McPherson G. L. and Meyerson S. L. (1991) Chern. Phys. Lett. (April) p. 325; Oomen et al: (1990) J. Luminescence 46: 353; NI Hand Rand SC (1991) Optics Lett. 16 (September); McFarlane R. A. (1991) Optics Lett. 16 (September); Koch et al. (1990) Appl. Phys. Lett. 56: 1083; Silversmith et al. (1987) Appl. Phys. Lett. 51: 1977; Lenth W. and McFarlane R. M. (1990) J. Luminescence 45:346; Hirao et al. (1991) J. Non-crystalline Solids 135: 90; McFarlane et al. (1988) Appl. Phys. Lett. 52:1300, incorporated herein by reference). In addition, methods of synthesizing submicron-diameter, monodispersed up-converting phosphor particles in a fluidized reactor bed are described in U.S. Pat. No. 6,039,894.

The up-converting phosphor particles used to form the layer on the carrier bead core are typically smaller than about 2 microns in diameter; for example, less than about 1 micron in diameter (i.e., submicron), or even 10 to 30 nanometers or less in diameter. In some embodiments, up-converting phosphor particles are as small as possible while retaining sufficient quantum conversion efficiency to produce a detectable signal. However, for any particular application, the size of the up-converting phosphor particle(s) to be used should be selected at the discretion of the practitioner. For example, the practitioner may select an up-converting phosphor particle size based on the desired bead core size and the degree of up-converting phosphor bead core loading. The practitioner may also consider the final external coating of up-converting phosphor-loaded bead when considering the size of an up-converting phosphor to use. The practitioner may also select the optimal size of up-converting phosphor particles on the basis of quantum efficiency data for the various up-converting phosphor particles of the invention. Such conversion efficiency data may be obtained from available sources (e.g., handbooks and published references) or may be obtained by generating a standardization curve measuring quantum conversion efficiency as a function of particle size.

The invention can be practiced with a variety of up-converting inorganic phosphors. For example, various embodiments of the invention employ one or more phosphors derived from one of several different phosphor host materials, each doped with at least one activator couple. Suitable phosphor host materials include, but are not limited to: sodium yttrium fluoride (NaYF₄), lanthanum fluoride (LaF₃), lanthanum oxysulfide, yttrium oxysulfide, yttrium fluoride (YF₃), yttrium gallate, yttrium aluminum garnet, gadolinium fluoride (GdF₃), barium yttrium fluoride (BaYF₅, BaY₂F₈), and gadolinium oxysulfide.

Suitable activator couples may be selected from, but are not limited to: ytterbium/erbium, ytterbium/thulium, and ytterbium/holmium. Other activator couples suitable for up-conversion may also be used. By combination of these host materials with the activator couples, at least three phosphors with at least three different emission spectra (red, green, and blue visible light) are provided. Generally, the absorber is ytterbium and the emitting center can be selected from: erbium, holmium, terbium, and thulium; however, other up-converting phosphors of the invention may contain other absorbers and/or emitters. The molar ratio of absorber: emitting center is typically at least about 1:1, more usually at least about 3:1 to 5:1, preferably at least about 8:1 to 10:1, more preferably at least about 11:1 to 20:1, and typically less than about 250:1, usually less than about 100:1, and more usually less than about 50:1 to 25:1, although various ratios may be selected by the practitioner on the basis of desired characteristics (e.g., chemical properties, manufacturing efficiency, absorption cross-section, excitation and emission wavelengths, quantum efficiency, or other considerations). The ratio(s) chosen will generally also depend upon the particular absorber-emitter couple(s) selected, and can be calculated from reference values in accordance with the desired characteristics.

The optimum ratio of absorber (e.g., ytterbium) to the emitting center (e.g., erbium, thulium, or holmium) varies, depending upon the specific absorber/emitter couple. For example, the absorber:emitter ratio for Yb:Er couples is typically in the range of about 20:1 to about 100:1, whereas the absorber:emitter ratio for Yb:Tm and Yb:Ho couples is typically in the range of about 500:1 to about 2000:1. These different ratios are attributable to the different matching energy levels of the Er, Tm, or Ho with respect to the Yb level in the crystal. For most applications, up-converting phosphors may conveniently comprise about 10-30% Yb and either: about 1-2% Er, about 0.1-0.05% Ho, or about 0.1-0.05% Tm, although other formulations may be employed.

Some embodiments of the invention employ inorganic UPT particles that are optimally excited by infrared radiation of about 950 to 1000 nm, preferably about 960 to 980 nm. For example but not limitation, a microcrystalline inorganic phosphor of the formula YF₃:Yb_{0.10}Er_{0.01} exhibits a luminescence intensity maximum at an excitation wavelength of about 980 nm. Inorganic phosphors of the invention typically have emission maxima that are in the visible range. For example, specific activator couples have characteristic emission spectra: ytterbium-erbium couples have emission maxima in the red or green portions of the visible spectrum, depending upon the phosphor host; ytterbium-holmium couples generally emit maximally in the green portion, ytterbium-thulium typically have an emission maximum in the blue range, and ytterbium/erbium usually emit maxi maximally in the green range. For example, Y_{0.80}Yb_{0.19}Er_{0.01}F₂ emits maximally in the green portion of the spectrum.

Although up-converting inorganic phosphor crystals of various formulae are suitable for use in the invention, the following formulae, provided for example and not to limit the invention, are generally suitable:

Na(YₓYb_{y}Er_{z})F₄: x is 0.7 to 0.9, y is 0.09 to 0.29, and z is 0.05 to 0.01.

Na(YₓYb_{y}Ho_{z})F₄: x is 0.7 to 0.9, Y is 0.0995 to 0.2995, and z is 0.0005 to 0.001.

Na(YₓYb_{y}Tm_{z})F4: x is 0.7 to 0.9, y is 0.0995 to 0.2995, and z is 0.0005 to 0.001.

(YₓYb_{y}Er_{z})O₂S: x is 0.7 to 0.9, Y is 0.05 to 0.12, and z is 0.05 to 0.12; and

(Y_{0.86}Yb_{0.08}Er_{0.06})₂O₃ is a relatively efficient up-converting phosphor material.

For example, but not to limit the invention, ytterbium(Yb)-erbium(Er)-doped yttrium oxysulfides luminesce in the green after excitation at 950 nm. These are non-linear phosphors, in that the ytterbium acts as an “antenna” (absorber) for two 950 nm photons and transfers its energy to erbium which acts as an emitter (activator). The critical grain size of the phosphor is given by the quantum yield for green emission and the doping level of both Yb and Er, which is generally in the range of about one to ten percent, more usually in the range of about two to five percent. A typical Yb:Er phosphor crystal comprises about ten to thirty percent Yb and about one to two percent Er. Thus, a phosphor grain containing several thousand formula units ensures the emission of at least one or more photons during a typical laser irradiation time. However, the nonlinear relationship between absorption and emission indicates that intense illumination at the excitation wavelength(s) may be necessary to obtain satisfactory signal in embodiments employing very small phosphor particles (i.e., less than about 0.3 μm). Additionally, it is usually desirable to increase the doping levels of activator/emitter couples for producing very small phosphor particles so as to maximize quantum conversion efficiency.

Inorganic microcrystalline up-converting phosphors with rare earth activators generally have narrow absorption and line emission spectra. The line emission spectra are due to f-f transitions within the rare earth ion. These are shielded internal transitions, which result in narrow line emission.

In certain applications, such as where highly sensitive detection is required, intense illumination may be provided by commercially available sources, such as infrared laser sources (e.g., continuous wave (CW) or pulsed semiconductor laser diodes). For example, in applications where the microcrystalline phosphor particle must be very small and the quantum conversion efficiency is low, intense laser illumination can increase signal and decrease detection times. Alternatively, some applications of the invention may require phosphor compositions that have inherently low quantum conversion efficiencies (e.g., low doping levels of activator couple), but which have other desirable characteristics (e.g., manufacturing efficiency, ease of derivatization, etc.); such low efficiency up-converting phosphors are preferably excited with laser illumination at a frequency at or near (i.e., within about 25 to 75 nm) an absorption maximum of the material. The fact that no other light is generated in the system other than from the up-converting phosphor allows for extremely sensitive signal detection, particularly when intense laser illumination is used as the source of excitation radiation. This unique property of up-conversion of photon energy by up-converting phosphors makes possible the detection of very small particles of microcrystalline inorganic phosphors. For practical implementation of up-converting phosphors as ultrasensitive labels, the grain size of the phosphor should be as small as practicable (typically less than about 0.3 to about 0.1 μm), for which laser-excited up-converting phosphors are well-suited.

For example, various phosphor material compositions capable of up-conversion which are suitable for the invention are shown in Table 1.

TABLE 1

| | Phospher Material Compositions | | |
|---|---|---|---|
| Host Material | Absorber Ion | Emitter Ion | Color |
| Oxysulfides (O [symbol] S) | | | |
| Y₂O₂S | Ytterbium | Erbium | Green |
| Gd₂O₂S | Ytterbium | Erbium | Red |
| La₂O₂S | Ytterbium | Holmium | Green |
| Oxyhalides (OX [symbol] ) | | | |
| YOF | Ytterbium | Thulium | Blue |
| Y₃OCI₇ | Ytterbium | Terbium | Green |
| Fluorides (F [symbol] ) | | | |
| YF [symbol] | Ytterbium | Erbium | Red |
| GdF₃ | Ytterbium | Erbium | Green |
| LaF₃ | Ytterbium | Holmium | Green |
| NaYF₃ | Ytterbium | Thulium | Blue |
| BaYF [symbol] | Ytterbium | Thulium | Blue |
| BaY₂F₆ | Ytterbium | Terbium | Green |
| Galiates(GaₓO_{y}) | | | |
| YGaO₃ | Ytterbium | Erbium | Red |
| Y [symbol] Ga [symbol] O₁₂ | Ytterbium | Erbium | Green |
| Silicates (SiₓO_{y}) | | | |
| YSi [symbol] O [symbol] | Ytterbium | Holmium | Green |
| YSi₃O [symbol] | Ytterbium | Thulium | Blue |

[symbol]

indicates data missing or illegible when filed

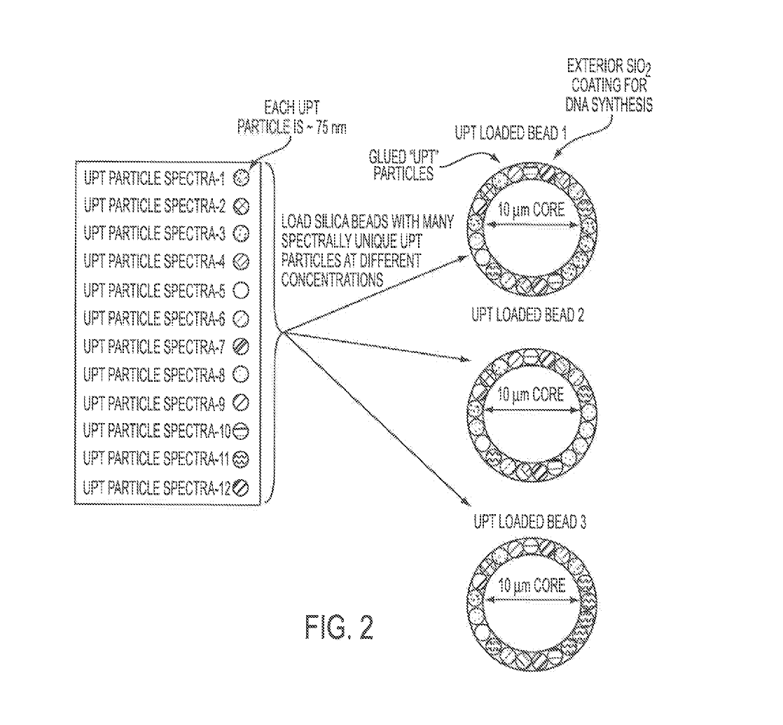

The numbers of up-converting phosphor particles per single carrier bead core are not limited in any way. Typically, as shown in FIG. 2, by changing the composition and concentrations of the up-converting phosphor particles on the surface of each bead, a large number of particles with distinguishable emissions properties may be obtained. In various embodiments of the invention, the number of unique combinations of up-converting phosphor-loaded beads may be increased by: (1) by increasing the number of up-converting phosphor particles with unique emission properties; or (2) by increasing the intensity resolution of the optical detector to enable accurate readout of different numbers of the same type of up-converting phosphor particle type loaded onto a single bead. The total number of combinations, N, of spectrally unique beads is given by the formula N═I^{m}, where I is the number of single up-converting phosphor particle intensity levels that can be resolved by the optical system, and m is the number of unique phosphor particles available for doping. For example, for four spectrally unique up-converting phosphor particles, and only two levels of optical resolution for each spectrally unique up-converting phosphor particle (either the up-converting phosphor particle is present on the bead or not present), there are a total of 16 possible combinations (2⁴). The total possible combinations of ten spectrally unique up-converting phosphor particles with four levels of optical resolution for each unique up-converting particle (not present, low intensity, medium intensity, and high intensity) would yield over one million combinations (4¹⁰). Table 2 shows the possible number of combinations of spectrally unique up-converting phosphor (UCP)-loaded beads given between one and twelve spectrally unique up-converting phosphor particles with between two and eight optical resolution levels. The column labeled “Colors” shows the number of unique up-converting phosphor particle spectra that can be generated. The “Levels” columns show the number of unique optical resolutions that can be achieved for each combination of up-converting phosphor particle spectra and resolution levels. The possible number of levels depends on the number of each unique up-converting phosphor-loaded bead the system can differentiate. The combinations highlighted in pink represent situations that result in >100,000 combinations; yellow boxes have >1 million combinations; and green boxes have >10 million combinations.

TABLE 2

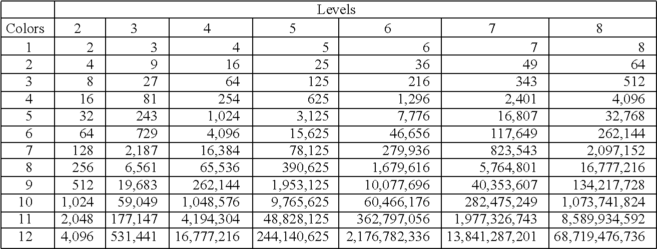

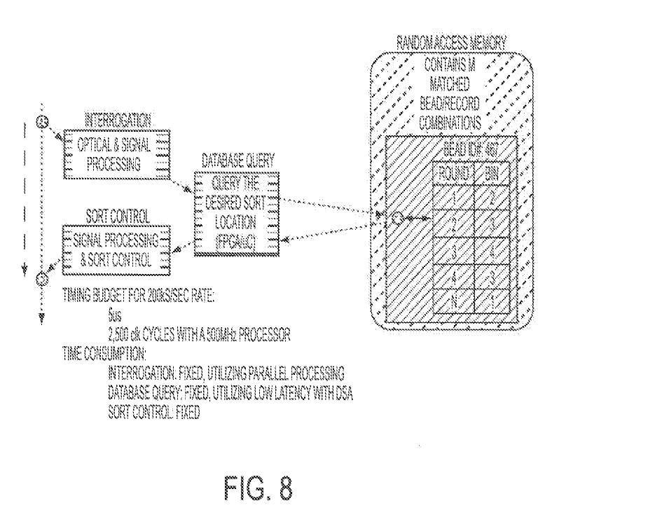

Up-converting phosphor-loaded beads, UCP-loaded beads, of the invention, may be fabricated by first well-dispersing the carrier beads and up-converting phosphor particles in alcoholic media. Dispersion can be achieved by well-known methods involving addition of dispersant and sonication. Next, a silica precursor is added to the dispersed beads and up-converting phosphor particles. The silica precursor is converted to silica via base catalyzed hydrolysis and condensation reactions. The silica binds to both component particles, and, by optimizing the relative amounts of the component particles, up-converting phosphor particle/carrier bead composite particles are formed without excessive agglomeration of like particles to themselves. In this preferred method the UCP particles themselves are preferentially coated initially with silica or glass. This may done as described or by other means known in the art. Thus, the attachment of the UCP particles to the bead has the advantage of being a silica to silica attachment rather than the attachment of differing chemical compositions to silica. This allows for attaching a number of potentially very different UCP particles in a single coating process and for creating a wide variety of and spectrally distinct UCP-loaded beads. FIG. 8 shows a graphical representation of the Dynamic Sorting Architecture (DSA) and the relationship between the high speed performance of the sorting method to the unit time window that enables that high speed. The DSA performs UCP-loaded bead identification, database query, and sort decision functions in immediate succession.

In various embodiments of the invention, UCP-loaded beads may also be encapsulated by an external coating, such as a ceramic coating (for example, silica). Encapsulation can increase the mechanical stability of up-converting phosphor-loaded beads beyond that achieved by the initial attachment process, and present a uniform chemical surface for subsequent attachment of chemical species. The surface of the UCP-loaded beads may be functionalized or coated to bind or link to a particular chemical species as a beginning reactant for the synthesis occurring in a particular method of the invention. Functionalizing or coating surfaces to improve their reactivity or to act as linkers for further chemistry is well-known in the art. The functional group or linker may also introduce the ability to cleave the product from the bead at the end of the method. That cleavage may be chemical, photochemical, or other means known in the art.

The encapsulation of UCP-loaded beads may be accomplished by the solution-based deposition of a ceramic precursor followed by heating, methods for which are well-established. In certain other embodiments of the invention, fluidized-bed coating methods may be used advantageously to deposit one or more ceramic or glass layers onto UCP-loaded beads. An advantage of the fluidized bed is that up-converting phosphor particles which are weakly attached to the carrier bead may be removed mechanically during fluidization, leading to a more stable UCP-loaded bead. In some embodiments of the invention, the UCP particles themselves are coated with a ceramic material, such as, but not limited to, silica, and then attached to the surface of a carrier bead. In certain other embodiments, the beads that are loaded with coated UCP particles are also encapsulated by an external coating as described above.

The total number of up-converting phosphor particles that can be attached to the surface of a bead core depends on the diameter of the particles and the diameter of the bead core. For example, in some embodiments of the invention, as few as five UPT particles on a single bead in combination with 3-color multiplexing can be detected. Emission intensity scales with up-converting phosphor particle volume. For example, for a 300 μm bead, the limit of detection may be equivalent to about 1,080 particles for 50 nm diameter particles, 320 particles for 75 nm diameter particles, and 135 particles for 100 nm.

Another parameter of the inventive method is the optical resolution level attainable in a flow cytometer for each unique up-converting phosphor particle. By increasing the number of levels of detection by the optical system of the flow cytometer, up-converting phosphor-loaded beads that were prepared using the same sized beads and particles can be further segregated by each level of detection based on the spectral characteristics of the loaded beads. Table 3 demonstrates this principle, and shows: 1) the number of particles between optical resolution levels for up-converting phosphor particles with 50, 75, and 100 nm diameters; and 2) bead cores with 5, 10, and 15 μm diameters using up-converting phosphor particles that belong to eight, ten, or twelve spectrally unique species of particles. The Table also shows four, six, and eight levels of detection for bead preparations containing eight, ten, or twelve spectrally unique species of particles. Therefore, the highlighted cell of Table 3 shows that 1,975 unique up-converting phosphor particles can be detected at each of four levels of detection, when 75 nm particles from twelve spectrally unique particle species are loaded on 10 μm diameter silica beads. Accordingly, a practitioner of the invention looking at the highlighted cell of Table 3 would understand that, by rounding 1975 to 2000, four levels of resolution can resolve approximately 0, 2000, 4000, and 6000 up-converting phosphor particles, respectively, based on increases of 2000 detection events per level. In other words, the number of up-converting phosphor particles per bead determines whether the system will decide if an intensity level is one, two, three, or four based on the ranges of up-converting phosphor particles per bead being either 0-2000, 2000-4000, 4000-6000, or 6000 (or more). The intensity level may be coded “1”, “2”, “3” or “4”, for example, by the computer system for a particular UCP wavelength. These are options for this component of the database identifier for a UCP wavelength.

TABLE 3

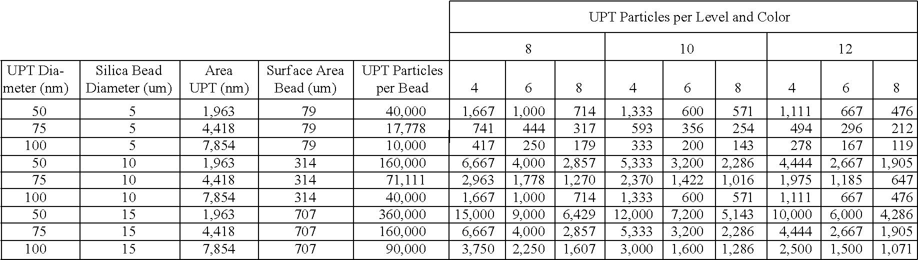

Accomplishing a low error rate in the detection system requires that the system be able to resolve detection of up-converting phosphor-loaded beads that contain a number of up-converting phosphor particles that falls between the levels of detection. In various embodiments, the method of the invention increases the number of levels of detection. In some embodiments, the method of the invention manufactures up-converting phosphor-loaded beads such that the number of phosphor particles per bead falls near the median number of particles that the optical system can detect at a given level of detection. By doing so, the method of the invention will minimize sorting errors. The minimum manufactured difference between beads in the number of UCP particles per bead should be a number greater than the detection resolution minimum for the optical sensors that detect UCP particle emissions. Such a requirement creates an optimally small detection error rate. For example, if a detection sensor can consistently determine the difference between the intensity of up-converting phosphor-loaded beads containing either 100, 200, or 300 phosphor particles per bead, then the up-converting phosphor-loaded beads should be prepared such that there is only allow a minimum difference of intensity between different beads of that contain approximately the same number of phosphor particles. Based on the foregoing example, the up-converting phosphor-loaded beads would ideally contain 150, 250, or 350 particles per bead, no beads would contain numbers of particles per bead on the borders between 150, 250, or 350 (i.e., 100, 200, or 300). By implementing such an approach to manufacturing up-converting phosphor-beads, the method of the invention avoids loaded beads with a numbers of particles that place them on the border between the levels of detection, which in turn, improves sort decision capability and thereby improves sort consistency performance.

Interrogation and Sorting of Up-converting Phosphor-Loaded Beads

As stated above, the invention relates to a method and platform that allows for the rapid, low-cost, parallel synthesis of custom chemical compounds and particularly polymers. The polymers can be of any type, wherein the monomeric subunits of the polymer are added by utilizing a chemical reaction. In various embodiments, the initial step in the polymer synthesis process takes potentially non-predetermined sets of UCP-loaded beads and detects and records their spectral identities into a database on a computer readable medium by using a computer system that is linked to a flow cytometer. The fluid stream of the flow cytometer may be reaction solvent system, a wash solvent, or a gas and different solvents may be used in each sorting or reaction step or in different bins of a method of the invention. The computer system controlling the flow cytometer can include a central processing device, an expandable memory, input devices such as an optical detection apparatus and the like, and output devices to steer the bead sort direction and human interface output devices such as a monitor, printer, computer storage device, and the like. The central processing device can be any high speed processor with parallel input channel processing capability and interface to a large memory addressing protocol, which can include the parallel configuration of multiple double data rate (DDR)2 or DDR3 memory components. Possible high speed processors include, but are not limited to an application-specific integrated circuit (ASIC), a field-programmable gate array (FPGA), or any other adequately fast microprocessor or microcontroller with the required number of input and output ports. The central processing device interfaces with random access memory space, which in most embodiments of the invention has fast latency and an expandable addressing space to accommodate large number of elements. Thus, the method of the invention may achieve quick access to large numbers of random accessed elements by reserving memory locations for all potential tag codes that may appear in the element population. For example, in an embodiment of the invention that includes the spectral characteristics of one billion up-converting phosphor-loaded beads, there would need to be one billion reserved locations. Each memory location associated with an individual bead's spectral characteristics contains the appropriate sequence building sort direction for each round of sorts that the flow cytometer performs. In a method of the invention at least one million UCP-loaded beads with unique spectral characteristics are sorted or re-sorted at a rate of at least fifty thousand UCP-loaded beads per second. As is known in the art, currently available sensor and sort hardware is capable of performing static sort decisions in the >70 k->100 k sorts per second range. With the low latency dynamic sorting method which may be used in the invention the dynamic sorting will not act as a bottleneck when practicing a method of the invention. In other words, the dynamic sorting architecture used in a method of the invention will not limit the sorting rate nor the number of elements held in the database. The sorting rate may only be limited by the sensor hardware limitations or the physical limit of UCP signal that can be emitted in the time window of a fast sort decision. Methods of the invention may also employ an expandable large memory addressing platform and quick bead ID to memory address translation.

In various embodiments of the invention, the central processing device also executes computer readable instructions from configuration memory to control the flow cytometer method. For example, a microcontroller can use software, and a FPGA can use firmware configuration, and an ASIC can have hard-coded state machines to control the flow cytometer method. The microcontroller can control include the flow cytometer's interrogation and multiparametric analysis of the beads, the driving of sort control to direct beads, and the analysis and database query to decide on the correct sort path for each bead at the current state of the sort procedure. The central processing device can also execute instructions from the memory to conduct multiparametric analysis of the physical and chemical characteristics of the particles. For example, the central processing device can execute instructions from the memory to direct a laser light onto the fluid stream of the flow cytometer, and receive the parametric characteristics of the up-converting phosphor-loaded beads using a fluorescent detector or detectors by configuring the detector(s) to receive a combination of scattered and fluorescent light and to analyze the changes in brightness at each detector. In turn, the central processing unit assigns an identification tag to each UCP-loaded bead by using a parallel combination of sensed parameters from the bead. For example, an UCP-loaded bead may have a parallel combination of spectral intensity over different spectral regions sensed from the particles on the bead.

Other aspects of the spectral output of a UCP tagging method may also be used to discern the tag. In various embodiments of the invention, the tag may be represented as a numerical concatenation of the individual sensed parameters to define the tag code. Such an identification tag may be optimally used directly as a memory address to quickly access a memory location corresponding to this tag without additional time consuming processing on the identification tag. In addition, the foregoing random memory accessing and search scheme will have optimal constant access time invariant to the size of UCP-loaded bead space. This sort decision and database query scheme enables the high speed sorting of an arbitrarily large number of unique UCP-loaded beads.

Random UCP-loaded beads become cataloged with predefined polymer sequence sort paths in a first step of a sort run in a method of the invention. The method of the invention dynamically matches the sequence from a user provided computer readable file with the up-converting phosphor-loaded bead tag in the large memory space. Up-converting phosphor-loaded beads identified with the same tag will have the same sorting sequence. The processing scheme can track the number of UCP-loaded beads that have been processed in a sort run that have the same tag. A method may also start with UCP-loaded beads which have been pre-functionalized and/or pre-sorted and their tag information loaded into the database.

Once the unique tags of the UCP-loaded beads have been matched with respective polymer sequences in the large memory space, the synthesis process can begin. Described here as relating to polymer synthesis, the method of the invention uses flow cytometry to sort each UPT-loaded bead into one of a number of bins depending on the first monomeric subunit of the polymer sequence that has been predefined for that bead. Thus, the number of bins generally reflects the number of different [types] of monomeric subunits that a particular polymer may comprise. For example, if the first monomeric subunit in the polymer sequence to be synthesized on a bead with a spectral identity of 1 is X, then that bead type will be sorted into the X bin by the flow cytometer in the first sort based on the spectral identity of the bead. Upon being sorted to the appropriate bin, the method of the invention couples a monomeric subunit that is correlated with the to the bead surface. The method of the invention then pools all of the beads from the bins and re-sorts the beads to the bins based on the next monomeric subunit in each respective bead's polymer sequence. Subsequent sorting runs use the spectral tag and preloaded sort sequence to sort an up-converting phosphor-loaded bead into the correct bin. The process is repeated until the entire desired sequence is synthesized on each bead.

Generally, in each sorting run, an UCP-loaded bead will go through an optical interrogation phase, and the resulting information will be used in a database query phase to determine in a sort control phase, the direction to sort the UCP-loaded bead in order to be sorted into the correct bin.

In various embodiments of the invention, the optical interrogation phase will uniformly excite an interrogated UCP-loaded bead to generate the most consistent “sort run to sort run” consistency in the emitted signal. Uniform excitation will surround the bead with infared (IR) light to compensate for any sort run to sort run-inconsistent three-dimensional positioning of the bead within the excitation chamber. With respect to sort-run consistency, the method of the invention can use redundancy to reduce inconsistency of the emitted signals from different sort runs. The invention also optimizes lag time between excitation and emission to generate the strongest and most consistent signal.

Typically, there are at least three parameters that may determine how a flow cytometer works within the context of the invention: The sort rate, which is the number of beads sorted per second; and Yield, which is the percentage of beads that are sorted in a single run (Unsorted bins may be sorted to waste or a recycle bin for subsequent sorting); and Bin purity, which is the percentage of beads in a bin that are supposed to have been sorted to the bin.

Generally, the sort rate and the yield are related to the concentration of the beads in solution. The higher the concentration of beads in solution, the more drops will have more than 1 bead; whereas the lower the concentration of beads in solution, the more drops will have zero beads. The yield is the number of drops that have only 1 bead (yield is dependent on the bead concentration and overall drop formation rate). The distribution of the number of beads in each drop is determined by Poisson statistics.

In various embodiments, the overall system yield is at least, or greater than 99.9%. In some embodiments, if the flow cytometer yields about 80%, it may be necessary to sort the beads collected in a recycle bin multiple times before proceeding to the next step of monomeric subunit addition in order to approach a yield rate of 99.9%.

According to Table 4, at 70% yield, 70% of the droplets with a bead would have only one up-converting phosphor-loaded bead, which means that 30% of the drops would have more than one bead. The sort rate, as determined by Poisson statistics, gives the number of drops that have one bead, assuming a droplet rate of 200,000 drops/second. Therefore, 33% of the drops would have zero beads at a 70% yield, 37% of the beads would have one bead, and 30% would have greater than one bead. The required sort for each yield case is the number of sorts necessary to yield 99.9% of the beads passing on to the next synthesis step.

TABLE 4
Total Beads 1.00E+07
Oligo Lengths 50
Droplet Rate 200,000

| | | | | Time for Individual Sorts (secs) | | | | | | |
|---|---|---|---|---|---|---|---|---|---|---|
| | Sort Rate | Req'd | Synthesis | Sort Time | | | | | | |
| Yield | (sorts/sec) | Sorts | Time (hrs) | (min) | Sort 1 | Sort 2 | Sort 3 | Sort 4 | Sort 5 | Sort 6 |
| 70% | 73,576 | 6 | 6.03 | 3.23 | 135.91 | 40.77 | 12.23 | 3.67 | 1.10 | 0.33 |
| 80% | 72,309 | 5 | 5.73 | 2.88 | 138.30 | 27.66 | 5.53 | 1.11 | 0.22 | N/A |
| 90% | 62,667 | 3 | 5.79 | 2.95 | 159.57 | 15.96 | 1.60 | N/A | N/A | N/A |
| 95% | 49,874 | 3 | 6.26 | 3.52 | 200.51 | 10.03 | 0.50 | N/A | N/A | N/A |

The total synthesis time is the required time (including both sorting and phosphoramidite chemistry) to synthesize a 50-mer library with 10 million different beads. The sort time is the total time required to sort the beads to 99.9% yield in the different per-step yield cases. One assumption for the sorting is that there is not a significant dilution of the beads on each subsequent sort. Overall, Table 4 shows that there is a trade between yield and sort rate.

The flow cytometer metric, purity, refers to the number of beads sorted to their correctly assigned bins. For example, the current BD Biosciences flow cytometer specification for purity is >98%, with respect to sorting cells. Because the UCP-loaded beads of the invention are more uniform than cells, the purity in most embodiments of the invention will be higher for up-converting phosphor-loaded beads than for cells. At 98% purity, the synthesis of a 50-monomeric subunit polymer from four distinct monomeric subunits (a four-bin sort) would result in only 36% of the beads having the correct final polymer sequence based on a binomial distribution of errors. Alternatively, method of the invention can achieve 99.96% purity if each bin is sorted twice, assuming the sorting purity is an independent event. Table 5 shows the percentage of beads with a given number of synthesis errors at the end of a 50-mer oligonucleotide synthesis that would have zero, one, two, three, four, and five sequence errors. The BD Influx® (BD Biosciences) sorts cells at a 98% rate of purity. In Table 5, the highlighted row shows an overall purity of 99.96%, assuming a 98% per step purity when the beads are twice sorted to each bin.

TABLE 5

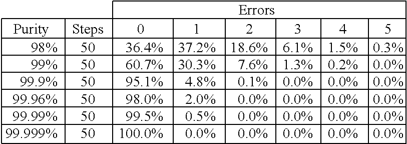

Chemical Synthesis and Polymer Fabrication

A sorting bin may be any container, vessel, or the like. For example, in some embodiments of the invention, the bin may be the well of a micro-titer plate; while in other embodiments, the bin may be a reaction chamber, such as a borosilicate or plastic tube. In various embodiments, the bin assignment for each up-converting phosphor-loaded bead is recorded on the computer-readable medium that is linked to the flow cytometer. The bin assignment information for each UCP-loaded bead may be saved in a database on the computer-readable medium that is linked to the flow cytometer. In some embodiments the computer-readable medium may be part of a computer system that is directly linked to the flow cytometer, while in other embodiments, the computer system may be remotely linked to the flow cytometer.

The chemical reactions that attach each of the monomeric subunits together to form polymers may occur directly in the bins, or alternatively, the reactions may take place in other locations. In some methods, the chemical reaction may be done with the pooled beads. A reaction step, may also include a washing step or, for a particular set or sets of UCP-loaded beads in the method, a holding step while other sets of beads are undergoing a particular reaction.

As mentioned above, any number of various methods of attaching a first monomeric subunit to the surface of a bead are available. Such methods are known in the art and depend on the particular chemical synthesis being performed in the method. For example, nucleic acid synthesis on the surface of an up-converting phosphor-loaded bead can be initiated by coupling of a DMT-protected phosphoramidite linker molecule to the surface of the bead. In some other embodiments of the invention, the surface of an UCP-loaded bead may be aminated with aminopropyltriethoxysilane for the purpose of attaching amino groups, however other omega-functionalized silanes can be substituted to attach alternative functional groups.

With respect to the reaction that adds a monomeric subunit to the monomeric subunit attached directly to the surface of the bead, it will be dependent on the type of polymer that is being assembled. For example, individual nucleic acids are linked together by phosphoramidite chemistry to form nuleic acid polymers. Polypeptide polymers are formed when amino acids are linked together by reactions to form peptide bonds between each amino acid in the polymer. More complex biological polymers, such as, but not limited to, actin fibers carbohydrates, peptoids, and peptide nucleic acids can also be assembled by the method of the invention.

In various embodiments, the sequence of monomeric subunits in a polymer sequence may be predetermined, while in other embodiments, the sequence may be random. In the case of a pre-determined polymer sequence, the sequence of monomers may be programmed into the computer system that is linked to the flow cytometer. For example, the sequence information may be entered in to the computer-readable medium that also contains the spectral identities of the up-converting phosphor-loaded beads. Accordingly, the polymer sequence may be entered into a database that correlates each polymer sequence with a particular up-converting phosphor-loaded bead based on its spectral characteristics.

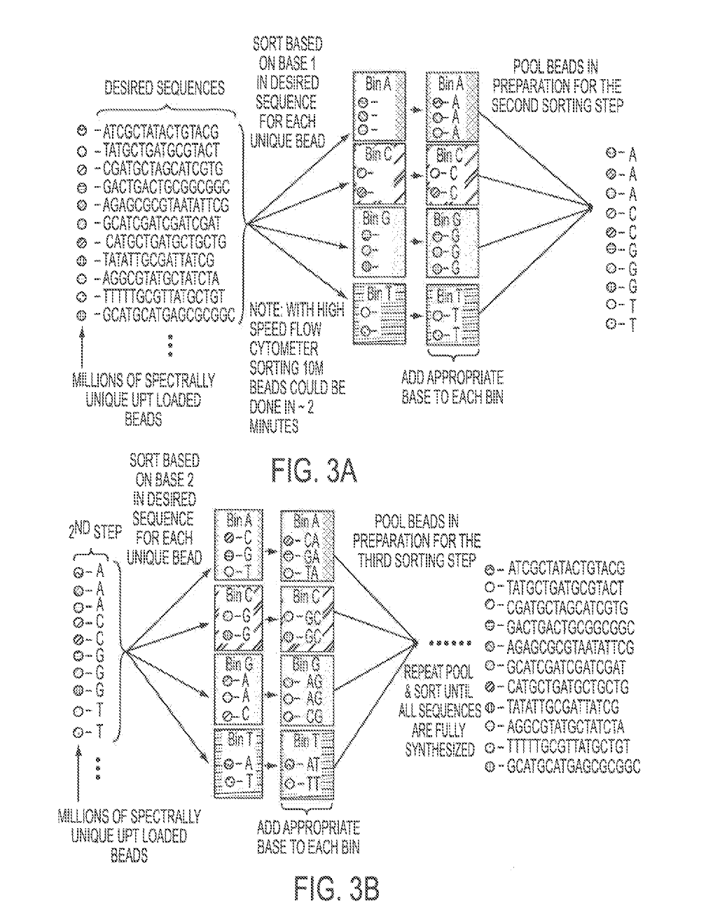

As discussed above, in various embodiments of the invention, DNA polymers may be synthesized by the method of the invention. A potential application of DNA polymer synthesis according to the invention may be the fabrication of DNA microarrays. Typically, for embodiments of the invention that relate to microarrays, each unique DNA sequence is synthesized onto spectrally unique up-converting phosphor-loaded silica beads in parallel using standard phosphoramidites. The initial step in the process takes a potentially non-predetermined set of up-converting phosphor-loaded beads and records their spectral properties using the flow cytometer. Once the spectral properties, also known as the spectral tags, are recorded for each bead into the database, the synthesis sorting process can begin. Each up-converting phosphor-loaded silica bead is sorted into either a bin for adenine (A), guanine (G), thymine (T), or cytosine (C), depending on the first base in the DNA sequence for that bead. For example, if the first base in the DNA sequence to be synthesized on a bead with spectrum one is A, then that bead type will be sorted into the A bin by the flow cytometer in the first sort. The appropriate DNA base is coupled to every bead either directly in each of the four bins, or, alternatively, the coupling reaction can occur in another location. Following the coupling reaction, all of the beads from the four bins are pooled together and re-sorted back into the original four bins based on the second base in each unique beads sequence. The process is repeated until the entire sequence is synthesized on each base. For example, the sequence ACCT would be sorted into the A bin on the first step, pooled and then sorted into the C bin on the second step, pooled and then sorted into the C bin again on the third step, and finally pooled and sorted into the T bin on the fourth step. In the first sort step, the A bin would include the example bead in addition to every other bead that had adenosine as the first base. FIG. 3 outlines the process of synthesizing a million different sequences using the method of the invention.

In addition to microarrays, the synthesis of nucleic acid polymers by the method of the invention also has other applications. Although such applications are only limited by a practitioner's imagination, in various embodiments, the method of the invention can generate DNA libraries that would be useful as a starting point for synthetic gene and genome construction.

As indicated above, the method of the invention involves a combination of up-converting phosphor and flow cytometric technologies. The method of the invention employs a flow cytometer hydrodynamically focus up-converting phosphor-loaded beads in a fluid flow through a flow cell, where the beads are interrogated by a laser. Based on the parameters of interest, each of the beads can be sorted into different bins. Typically, flow cytometers are used to sort cells based on size and the binding of fluorescent dyes. The BD Influx®, from BD Biosciences, has the ability to sort samples at rates as high as 200,000 events per second. The sorter is capable of the analog/digital conversion of the signals from up to 16 different channels (typically 14 different color channels and two scatter channels) at this rate.^{[7]}

Each different polymer sequence is synthesized onto spectrally unique UPT-loaded silica beads in parallel. As an initial step in the process may take a potentially non-predetermined set of UPT-loaded beads and records their spectral identity using the flow cytometer. Once the spectral tag for each bead is loaded into the database, the synthesis sorting process can begin. Each UCP-loaded bead is sorted into one of a number of bins depending on the first monomeric subunit of the polymer for that bead. Then, once the monomeric subunits are attached to the beads, the beads are pooled, and resorted into bins according to the next monomeric subunit in the sequences of each respective bead. The steps of pooling, attachment, and sorting are repeated until the polymers reach the desired length. A non-limiting example of the method of the invention applied to the synthesis of DNA polymers could occur as follows. If the first base in a DNA sequence to be synthesized on a bead with spectrum 1 is adenosine (A), then that bead type will be sorted into the A bin, rather than into one of the bins of the other three nucleotide bases of DNA, thymine (T), guanine (G), or cytosine (C), by the flow cytometer in the first sort. The appropriate DNA base is coupled to every bead in each of the four bins. Then all of the beads in the four bins are pooled together and re-sorted back into the original four bins based on the second base in each unique beads sequence. The process is repeated until the entire sequence is synthesized on each base. In other words, the sequence ACCT would be sorted into the A bin on the first step, pooled and then sorted into the C bin on the second step, pooled and then sorted into the C bin again on the third step, and finally pooled and sorted into the T bin on the fourth step. In the first sort step, the A bin would include the example bead in addition to every other bead that had adenosine as the first base. FIG. 3 outlines the process of synthesizing a million different sequences using the flow cytometry-UPC approach of the invention.

More specifically, FIG. 3 shows a graphical representation of the first two steps necessary to fabricate a DNA microarray on the UCP-loaded beads. The first step of the process is to determine what sequence will be added to each spectrally unique UCP-loaded bead. The flow cytometer is then used to sort the beads into bins based on the first base in the desired sequence. Next, the base is added and the beads are re-pooled. The end result is a pool of UCP-loaded beads with the first base in the desired sequence added. Next, the UCP-loaded beads are resorted, based on the second base in the desired sequence for each bead. The appropriate base is added to all the beads in each bin, and then the beads are re-pooled for the third step. The process is repeated until the desired sequence is fully synthesized on each UCP-loaded bead.

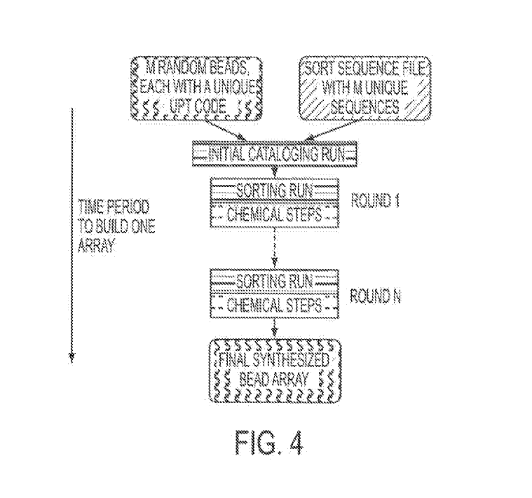

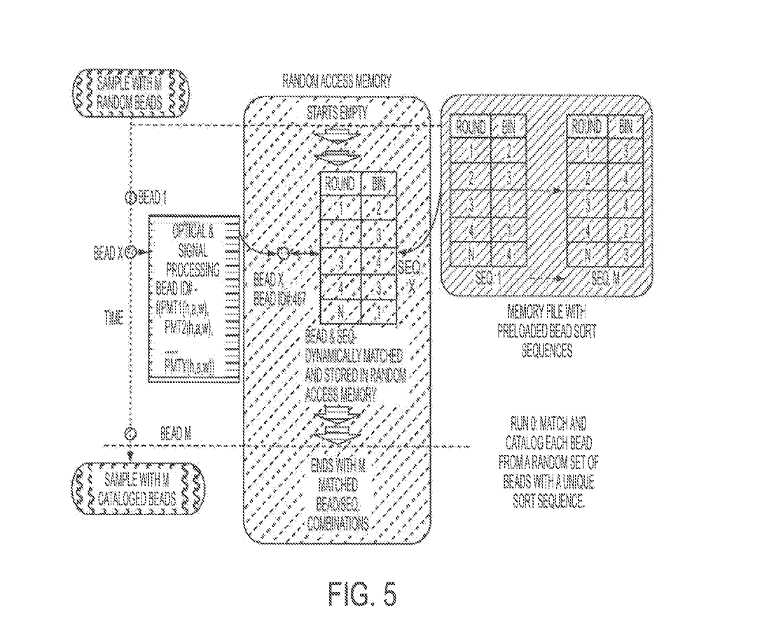

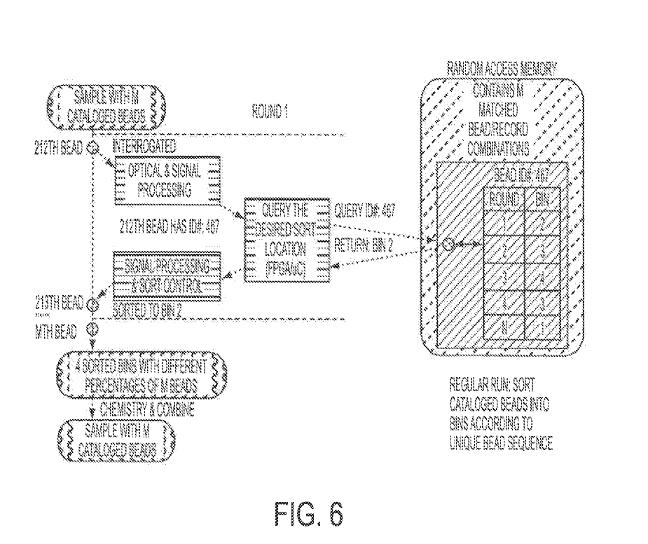

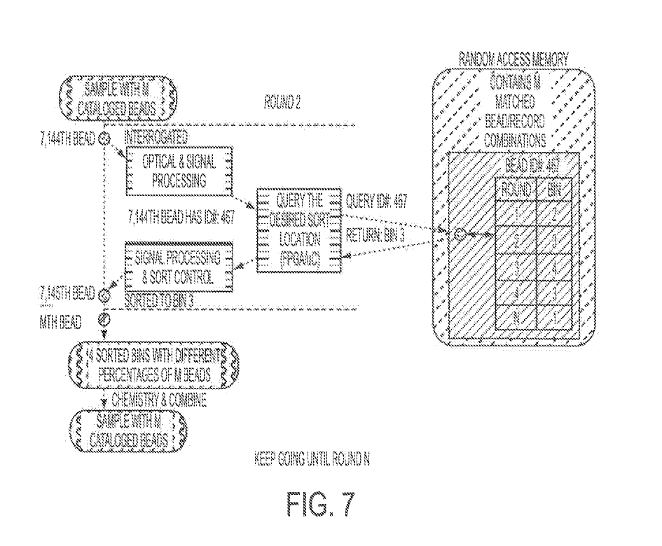

FIG. 4 shows a graphic representation of a dynamic sorting architecture example for in DNA synthesis using the method of the invention. FIG. 5 shows a more detailed graphical representation of the initial catalog run of UPC-loaded particles that FIG. 4 depicts. FIG. 6 shows a more detailed graphical representation of the first run (Round 1) that FIG. 4 depicts. FIG. 7 shows a more detailed graphical representation of the second run (Round 2) that FIG. 4 depicts.

Moreover, in other embodiments, the method of the invention may be used to form polymers of amino acid residues, such as peptides, polypeptides and proteins. With such polymers in mind, the surface of an UCP-loaded bead can be aminated for the purpose of attaching amino groups, for example, by treating the surface with aminopropyltriethoxysilane. Other omega-functionalized silanes can be substituted to attach alternative functional groups.

However, regardless of application, the method of the invention is efficiently repeatable to allow the production of multiple bead sets, and by extension, multiple sets of polymers. The exact spectral characteristics of beads going into a set do not need to be identical for each produced set of beads. In some embodiments, a random picking process can grab a statistically determined large enough set of beads from a pool of unique beads as long as the beads mix in a correct randomized manner.

As discussed above, the methods of the invention may be used in any chemical synthesis where sequential steps are used to prepare chemical compounds. The methods are particularly useful for the rapid, parallel synthesis of compounds where variations can be introduced in parallel. Thus, for example, the methods of the invention may be used to produce vinyl block co-polymers having varying lengths of the particular blocks. Or, the methods may be used to produce polyesters having, for example, the same alcohol component(s) and different acid components. Block polyethers with differing ether monomeric units may also be produced using the methods of the invention. The methods of the invention may also be used to sort and introduce separate functional groups into a common starting material. Derivatives of small molecules having a common core structure may also be produced where the common core structure is first synthesized on all UCP-loaded beads and then the further individual derivatization is accomplished in subsequent steps in a method of the invention. Generally speaking, the methods of the invention use known synthetic steps and chemical to prepare variations of a compound in parallel and accomplishing individual reaction steps in rapid succession by sorting through flow cytometry and the use of UCP-loaded beads as the carrier/reaction substrate.

### EXAMPLES

### Example 1

In the following example, approximately 6 to 6.5 micron diameter silica particles were coated with YYbEr particles.

One gram of silica particles was added to a bottle containing 50 g of anhydrous ethanol. The mixture was sonicated for 0.5 hr in a Branson® (Danbury, Conn.) model 1210 ultrasonic bath, and then magnetically stirred at 500 rpm for 16 hr to disperse the silica particles (the silica dispersion)

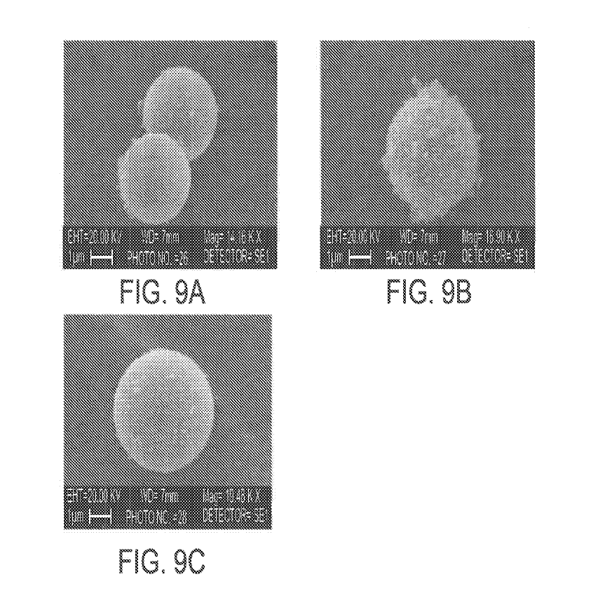

In a separate bottle, 1.0 g Disperbky®-190 (BYK Additives & Instruments, the Altana Group, Wesel, Germany) was dissolved in 50 g anhydrous ethanol, and then 0.1 g of YYbEr particles was added to the solution. The mixture was sonicated for 0.5 hr, and magnetically stirred at 500 rpm for 16 hr to disperse the YYbEr particles (the up-converting phosphor dispersion). The up-converting phosphor dispersion was added, dropwise with a pipette, to the silica dispersion, and the mixture was sonicated for 0.5 hr, and then magnetically stirred for three days to make a silica/up-converting phosphor dispersion. Following the stirring step, a solution containing 1.25 g of tetraethyl orthosilicate in 5 g of anhydrous ethanol was added dropwise to the silica/up-converting phosphor dispersion, and the dispersion was magnetically stirred for 1.5 hr. Then 4 g of concentrated NH₄OH in 10 g of anhydrous ethanol was added dropwise over the course of 40 min to the silica/up-converting phosphor dispersion. The dispersion was then magnetically stirred at 500 rpm for 16 hr. Afterwards, the dispersion was filtered through a 1.2 μm Whatman GF/A filter (Whatman, Ltd., Kent, UK). The product left on the filter was washed with water and isopropanol, and then dried at 55° C. for 2 hr. FIG. 9 shows scanning electron micrographs of the product.

### Example 2

In the following example, approximately 6 to 6.5 micron diameter silica particles were coated with coated with three different up-converting phosphor particles.

One gram of silica particles were added to a bottle containing 50 g of anhydrous ethanol. The mixture was sonicated for 0.5 hr in a Branson® (Danbury, Conn.) model 1210 ultrasonic bath, and then magnetically stirred at 500 rpm for 16 hr to disperse the silica particles (the silica dispersion)

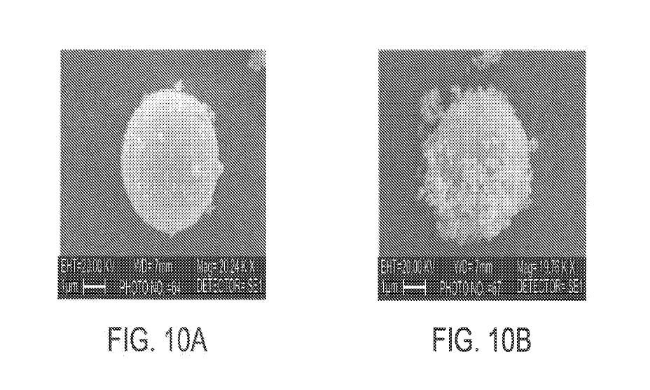

In three separate bottles, 0.5 g Disperbky®-190 (BYK Additives & Instruments, the Altana Group, Wesel, Germany) was dissolved in 25 g anhydrous ethanol, and then 30 mg each of three different species of up-converting phosphor particles were added to the three solutions. The up-converting phosphor particles emitted green, blue, and yellow light respectively. The dispersions were sonicated for 0.5 hr, and magnetically stirred at 500 rpm for 16 hr to disperse the up-converting phosphor particles (the up-converting phosphor dispersions). The up-converting phosphor dispersions were added, dropwise with a pipette, to the silica dispersion, and the mixture was sonicated for 0.5 hr, and then magnetically stirred for one hour to make a silica/up-converting phosphor dispersion. Following the stirring step, a solution containing 1.25 g of tetraethyl orthosilicate in 5 g of anhydrous ethanol was added dropwise to the silica/up-converting phosphor dispersion, and then the dispersion was magnetically stirred for 1.5 hr. Afterwards, 4 g of concentrated NH₄OH in 10 g of anhydrous ethanol was added dropwise over the course of 40 min to the silica/up-converting phosphor dispersion. The dispersion was then magnetically stirred at 500 rpm for 16 hr. Next, the dispersion was filtered through 1.2 μm Whatman GF/A filters (Whatman, Ltd., Kent, UK). The product left on the filter was washed with water and isopropanol, and dried at 55° C. for 2 hr. FIG. 10 shows scanning electron micrographs of the product.

### REFERENCES

1. Cello, J., A. V. Paul, and E. Wimmer, Chemical synthesis of poliovirus cDNA: Generation of infectious virus in the absence of natural template. Science, 2002. 297(5583): p. 1016-1018.
2. Smith, H. O., et al., Generating a synthetic genome by whole genome assembly: phi X174 bacteriophage from synthetic oligonucleotides. Proceedings of the National Academy of Sciences of the United States of America, 2003. 100(26): p. 15440-15445.
3. Gibson, D. G., et al., Complete chemical synthesis, assembly, and cloning of a Mycoplasma genitalium genome. Science, 2008. 319(5867): p. 1215-1220.
4. Martin, V. J. J., et al., Engineering a mevalonate pathway in Escherichia coli for production of terpenoids. Nature Biotechnology, 2003. 21(7): p. 796-802.
5. Hutchison, C. A., et al., Global transposon mutagenesis and a minimal mycoplasma genome. Science, 1999. 286(5447): p. 2165-2169.
6. Posfai, G., et al., Emergent properties of reduced-genome Escherichia coli. Science, 2006. 312(5776): p. 1044-1046.
7. BD_Biosciences, BD Influx Technical Specifications. 2009. p. 1-4.

1. A carrier bead having a generally spherical shape and a layer of at least one up-converting phosphor particle on the bead's surface.

2. A bead according to claim 1, wherein the bead has a metallic layer between the bead surface and the up-converting phosphor particle layer.

3. A bead according to claim 1, wherein the bead is a ceramic bead.

4. A bead according to claim 1, having an external coating encapsulating the bead and up-converting phosphor particle layer.

5. A bead according to claim 4, wherein the external coating is a silica coating, a glass coating, or a ceramic coating.

6. A bead according to claim 1, wherein the up-converting phosphor particle layer comprises at least two up-converting phosphor particles having distinct emission wavelengths.

7. A bead of claim 1, wherein the diameter of the bead core is any diameter up to about 20 μm.

8. A bead of claim 7, wherein the up-converting phosphor particles have a diameter of at least 50 nm, at least 75 nm, at least 100 nm, or at least 300 nm.

9. A bead according to claim 8, having an external coating encapsulating the bead and up-converting phosphor particle layer.

10. A bead according to claim 9, wherein the external coating is a silica coating, a glass coating, or a ceramic coating.

11. A method of synthesizing at least two polymers by a stepwise combination of monomeric units, wherein the method comprises the steps of:
a) providing at least two sets of UCP-loaded beads, wherein the UCP-loaded beads within each set are spherical beads with a layer of at least one up-converting phosphor particle on the bead surface and each set has a unique excitation or emission identity;
b) optionally attaching a monomeric subunit to the at least two sets of UCP-loaded beads;
c) detecting the emission properties of the UCP-loaded beads using a computer system-controlled flow cytometer;
d) recording the emission properties of each set of UCP-loaded beads to a database located on a computer-readable medium using the computer-controlled flow cytometer, wherein the database assigns each unique UCP-loaded identity to a specified polymer sequence;
e) sorting the UCP-loaded beads into any one of a number of bins by sets, wherein each bin is correlated with a specified monomeric subunit, and wherein the assignment of each UCP-loaded bead to a bin is based on the first monomeric subunit of the polymer sequence that is assigned to the UCP-loaded bead in the database of step (d);
f) attaching the monomeric subunits within each bin to the surfaces of the UCP-loaded beads sorted to the bin;
g) pooling the UCP-loaded bead sets after completion of step (e)
h) optionally re-sorting the UCP-loaded beads from step (f) into bins using the computer-controlled flow cytometer, wherein the UCP-loaded beads' spectral identities are detected, and each UCP-loaded bead is sorted to a bin according to the next monomeric subunit to be added to the polymer sequence assigned to each UCP-loaded bead set in the database of step (c);
i) reacting the UCP-loaded beads under conditions sufficient to attach a selected monomeric subunit to the most-recently attached monomeric subunit;
j) pooling the UCP-loaded beads after completion of step (h);
k) repeating steps (g)-(i) to produce a desired polymer on each set of UCP-loaded beads; and
l) optionally cleaving a polymer from its UCP-loaded bead.

12. The method according to claim 11, wherein the sorting step comprises:
illuminating at least two UCP-loaded beads with excitation radiation;
detecting emission radiation of UCP-loaded beads; and
sorting the UCP-loaded beads to bins as described in step (d).

13. The method according to claim 11, wherein the step of attaching a monomeric subunit comprising absorbing the monomeric unit to the UCP-loaded surface or chemically reacting a monomeric subunit to UCP-loaded bead surface.

14. The method of claim 11, wherein the reacting step (i) occurs with the pooled UCP-loaded bead sets of step (g) or in one or more bins with the re-sorted beads of step (h).

15. The method according to claim 11, wherein steps (d) and (e) are performed using a low latency database building and query scheme architecture.

16. The method according to claim 11, wherein at least one million UCP-loaded beads with unique spectral characteristics are sorted or re-sorted at a rate of at least fifty thousand UCP-loaded beads per second.

17. The method according to claim 11, wherein the computer system-controlled flow cytometer comprises optical interrogation sensors, analog to digital signal conversion, high-speed digital signal processing, expandable parallel addressable memory, and sort direction control.

18. The method according to claim 14, wherein the at least two polymers produced are nucleic acid polymers having different nucleic acid sequences.

19. The method according to claim 18, wherein the nucleic acid polymers are DNA polymers.

20. The method according to claim 18, further comprising the step of forming a microarray of the UCP-loaded beads carrying the produced polymer.

21. The method according to claim 18, further comprising the step of forming an expression library of the UCP-loaded beads carrying the produced polymer.

22. The method according to claim 18, further comprising the step of forming a genomic library of the UCP-loaded beads carrying the produced polymer.

23. The method according to claim 18, wherein the method constructs a genome for a synthetic organism.

24. The method of claim 11, wherein the polymer is a peptide or a protein.

25. (canceled)

26. A method of claim 11, wherein the UCP-loaded beads in step (a) further comprise a functional coating to attach a monomeric unit to the beads.

27. A method stepwise chemical synthesis, wherein the method comprises the steps of:
a) providing at least two sets of UCP-loaded beads, wherein the UCP-loaded beads within each set are spherical beads with a layer of at least one up-converting phosphor particle on the bead surface and each set has a unique excitation or emission identity;
b) optionally attaching a first reactant to the at least two sets of UCP-loaded beads;
c) optionally detecting the emission properties of the UCP-loaded beads using a computer system-controlled flow cytometer;
d) recording the emission properties of each set of UCP-loaded beads to a database located on a computer-readable medium using the computer-controlled flow cytometer, wherein the database assigns each unique UCP-loaded identity to a specified product to be synthesized;
e) sorting the UCP-loaded beads into any one of a number of bins by sets, wherein each bin is correlated with a specified sequenced reaction step;
f) reacting the sorted UCP-loaded beads in step (e) according to the specified sequenced reaction step;
g) pooling the UCP-loaded bead sets after completion of step (f);
h) optionally re-sorting the UCP-loaded beads from step (g) into bins using the computer-controlled flow cytometer, wherein the UCP-loaded beads' spectral identities are detected, and each UCP-loaded bead is sorted to a bin according to the next sequential reaction step assigned to each UCP-loaded bead set in the database of step (d);
i) reacting the UCP-loaded beads in step (g) or step (h) according to the specified sequenced reaction step;
j) repeating steps (g)-(i) to synthesize a desired compound on each set of UCP-loaded beads; and
k) optionally cleaiving a desired compound from its UCP-loaded bead.

28. A method of making an up-converting phosphor loaded bead comprising the steps of:
dispersing carrier beads and up-converting phosphor particles in alcoholic media to form a dispersion,
adding at least one silica precursor is added to the dispersion, and
converting the silica precursor to silica via a base catalyzed hydrolysis or a condensation reaction.

29. A bead according to claim 1, wherein the carrier bead is a silica beed and the up-converting phosphor particles in the up-converting phosphor particle layer are silica-coated up-converting phosphor particles.
